(19) United States
(12) Patent Application Publication
(10) Pub. No.: US 2021/0220178 A1
(43) Pub. Date: Jul. 22, 2021

(54) STENT AND STENTING METHOD

(71) Applicant: DOYA MEDICAL LTD., Caesarea 3088900 (IL)

(72) Inventors: Galit AVIOR, Caesarea (IL); Tanhum FELD, Moshav Merhavya (IL)

(73) Assignee: DOYA MEDICAL LTD., Caesarea (IL)

(21) Appl. No.: 16/336,742

(22) PCT Filed: Sep. 26, 2017
(86) PCT No.: PCT/IL2017/051081

§ 371 Date: Mar. 26, 2019

Related U.S. Application Data
(60) Provisional application No. 62/399,674, filed on Sep. 26, 2016.

(51) Int. Cl. A61F 11/00; A61F 2/18

(52) U.S. Cl. CPC A61F 11/002; A61F 2/18; A61F 2250/0018; A61F 2230/0008; A61F 2002/183

(57) ABSTRACT

Provided is a stent for deployment in the Eustachian tube and other body passageways that supports the walls and assists in the natural opening of passage without hindering the natural closing operation.

### TECHNOLOGICAL FIELD

This disclosure concerns a stent, particularly useful for placement within the Eustachian tube as well as a stenting method for such placement.

### BACKGROUND ART

References considered to be relevant as background to the presently disclosed subject matter are listed below:
1. Bluestone, C D. Eustachian Tube Structure, Function, Role in Otitis Media. B C Decker Inc; Hamilton, Ontario: 2005
2. Bluestone, C D.; Klein, J O. Otitis Media in Infants and Children. B C Decker; Hamilton, Ontario: 2007
3. Stephen Chad Kanick and William J. Doyle, Barotrauma during air travel: predictions of a mathematical model. J Appl Physiol 98:1592-1602, 2005. First published Dec. 17, 2004
4. F. J. Sheer, J. D. Swarts, and S. N. Ghadiali, Three-dimensional Finite Element Analysis of Eustachian Tube Function under Normal and Pathological Conditions. Med Eng. Phys. 2012, 34(5): 605-616
5. Poe D S, et al. Analysis of Eustachian tube function by video endoscopy. Am J Otol. 2000; 21:602-607
6. International published patent application WO 2009/001358
7. U.S. Pat. No. 9,510,976
8. U.S. Pat. No. 6,589,286

Acknowledgement of the above references herein is not to be inferred as meaning that these are in any way relevant to the patentability of the presently disclosed subject matter.

### BACKGROUND

Adequate ventilation and drainage is essential for normal middle ear function, and it is the Eustachian tube (ET) that normally provides for that. Chronic Eustachian tube dysfunction has been implicated in the pathogenesis of many otologic disorders and is thought to be a principal cause of a variety of otologic surgical failures. Patients with chronic middle ear disease have often been shown to have a mechanical narrowing of the ET, usually at the isthmus (junction of the bony and cartilaginous portions). ET dysfunction has also been associated with functional disorders of the cartilaginous part.

The Eustachian tube is ordinarily closed in the resting position and dilates to the open position typically with swallowing, yawning, and with other voluntary or involuntary efforts. Tubal opening typically lasts less than one-half second. Closure of the Eustachian tube is maintained by a valve-like function of the opposing mucosal surfaces, submucosal tissue, fat, muscle, and cartilage. This natural valve measures approximately 5 mm in length and lies within the cartilaginous portion of the ET located about 10 mm distal into the tube from the nasopharyngeal orifice's posterior cushion or torus tubarius.

A common problem resulting from Eustachian tube dysfunction is Otitis Media with Effusion (OME) or the presence of fluid in the middle ear with no signs or symptoms of acute ear infection. Persistent middle ear fluid from OME results in decreased mobility of the tympanic membrane and serves as a barrier to sound conduction. OME may occur spontaneously because of poor Eustachian tube function or as a response following Acute Otitis Media. This may occur in infants and children ages 1-6 years due to anatomical difference and physiological changes of the Eustachian tube. At birth, the tube is horizontal and approx. 17-18 mm long. It grows to be at an incline of approx. 45 degrees and reaches a length of approx. 35 mm in adulthood. Due to its' relatively horizontal position in infants and young children and the relatively short length, these subjects are more likely to suffer from Eustachian tube dysfunction.

Most surgical procedures performed to treat these ontological conditions involve bypassing the blocked ET by implantation of a surgical prosthesis, usually in the tympanic membrane (ear drum), for ventilation of the middle ear cavity via the external ear canal. Tympanostomy tubes are recommended for initial surgery. Often, however, complications are encountered with such tubes. Early complications include: persistent otorrehea 10-26%, blockage of the tube 0-9%, early extrusion, and hearing loss. Late complications include: persistent perforation after tube extrusion 3%, scarring of the tympanic membrane, atrophic membrane 21-28%, granuloma 5-40%, Tympanosclerosis 40-65%, and cholesteatoma 1%.

US 6,589,286 and WO 2009/001358 disclose stents for deployment in the ET (“ET stent(s)”). While the former discloses such stent in general, that specifically disclosed and illustrated in the drawings of WO 2009/001358 has a leaf valve that enhances ventilation and drainage to the middle ear as well as a stenting method for inserting the stent in the ET.

An ET stent with a length-dependent radial strength to allow it to stay within the ET and allow normal closing and opening of the ET is disclosed in U.S. Pat. No. 9,510,976.

### GENERAL DESCRIPTION

Provided by this disclosure are stent, stent deployment assembly (comprising a stent, a stent delivery system and optionally an sent removal device) and a stenting method. The stent of this disclosure has a unique design that renders it suitable, although not exclusively, for deployment in the Eustachian tube (ET) and assist the natural opening of the ET without hindering the natural closing operation. Below in this disclosure, mention of an ET stent denotes an ET stent that embodies the features of a stent provide by this disclosure.

In addition to the use as an ET stent, a stent that embodies the characterizing features of this disclosure may be adapted to supplement and assist the natural opening operation of other variable body passageway, without hindering the natural closing operation. A stent of this disclosure may also, for example, be configured for deployment in the bile duct for the treatment of Sphincter of Oddi Dysfunction (SOD), in which the sphincter can malfunction, not letting the digestive juices through as it should; or configured for deployment in the esophagus for the treatment of lower or upper esophagus sphincter dysfunction; or configured for deployment in the urethra for the treatment of urethra sphincter dysfunction.

The stent of this disclosure is a collapsible stent which functions to open a blocked or clogged lumen and, at the same time, is adapted to permit the natural closing and opening of the lumen. For example, an ET stent is configured to support the walls of the ET to thereby facilitates drainage of fluids from the middle ear to the nasopharynx cavity and pressure equalization in the middle ear, while permitting closure of the natural valve of the ET.

By one embodiment the stent has a scaffold formed by struts that are shaped so as to form cells within the scaffold, which may be open or closed cells or a combination of the two. The properties of the struts (e.g. width, thickness, etc.) and cells' properties (e.g. size, whether the cells are open or closed cells or the relative proportion of open and closed cells) are all parameters that influence the scaffold's flexibility and rigidity. The scaffold is typically designed to have an oversize (namely the cross-sectional dimensions that the stent expands to if unhindered is larger than the cross-sectional dimensions of the lumen in which it is deployed) and the extent of the oversize may also be important in controlling the scaffold's flexibility or rigidity. These characteristics are typically achieved without any structural discontinuity of the struts.

It has also been realized, in accordance with some embodiments of this disclosure, that this function of opening a variable passageway such as the ET, on the one hand, and permitting natural closure on the other hand, can best be achieved by a stent which is not axial-symmetric but rather has a longitudinal plane of symmetry with two sides of the stent that are mirror images of one another. For example, the ET portion proximal to the nasopharynx cavity acts as a natural valve in that its walls can collapse to thereby close this passageway; and opened, through the contraction of surrounding smooth muscle, by moving away from one another. Opening occurs, for example, in response to actions of swallowing or yawning. The contraction in many sphincters in a tubular passageway, however, is not in an axial symmetric manner, but rather achieved through a concerted displacement of two opposite walls portions towards one another. The stent of this disclosure is, particularly, useful for deployment in tubular passageways in the body that have a sphincter, which operates to close and open the passageways in an axial non-symmetric manner

A Particular embodiment of a stent of this disclosure is, thus, a stent embodying the above features and having a longitudinal plane of symmetry (a plane defined and extending along the stent's axis), rather than an axial one. According to this embodiment, the stent has a peripheral scaffold that is intrinsically biased into an expanded state, which is a state in which the scaffold extends to its maximum extent. In this expanded state, the stent has a length defined between a proximal and a distal end of the scaffold. The stent's scaffold, as noted above, has a longitudinal plane of symmetry that extends lengthwise along the stent, between the two ends. In other words, the stent's scaffold, although not being axial-symmetric, has two side that are mirror images of one another.

It is a feature of the stent of this embodiment that an inwardly directed force, on the mirror-image sides, in a direction normal to the plane of symmetry, causes an inwardly directed displacement that is larger than that caused by the same inwardly directed force applied, however, in a direction parallel to said plane of symmetry. In other words, this stent reacts differentially to a radial force in different directions, exerting a weaker displacement resistance to a force applied in a direction normal to said longitudinal plane of symmetry as compared to that of a force applied in a direction normal thereto.

In the following description, the term “axial” is used to denote a general orientation defined by the stent's axis (extending between its two ends), although it is understood that once deployed, the stent may not be entirely straight and accordingly the axis may be curved; the term “vertical” is used to denote a direction is be used to denote a direction normal to the axis and normal to said plane of symmetry. Further, the terms “proximal” and “distal” are used in relation to the direction in which it is inserted and deployed; for example, in the case of an ET stent, the proximal end or segment is that closest to the nasopharynx cavity. The terms “lumen” or “passageway” are used to denote such structures within the body.

The stent of this disclosure will be occasionally described below in reference to an ET stent, this description intended to illustrate the teachings of this disclosure and is not intended in any way to be limiting.

In use, the ET stent is deployed such that its lateral scaffold portions come to bear on the lateral wall portions of the ET. Thus, when the ET lateral walls, particularly the proximal ones constituting the natural valve, they bear on and act upon the weaker portions of the scaffold wall. The lateral portions of the scaffold are configured to have elastic properties such that they expand with the lateral wall during the walls expansion and opening and provide also relatively small resistance that would not hinder the walls' collapse to seal the passageway. In other words, although the ET stent adds support to opening, it does not function cause the passageway to remain constantly open and, hence, facilitates natural valve function.

As noted, the stent of this disclosure is typically axial non-symmetric. Furthermore, the overall cross-section of the stent primarily in the proximal segment is non-circular and may, for example, be oval or elliptic.

The ET stent may be configured for deployment in only the proximal portion of the ET; or it may be configured for deployment in only in the cartilaginous part of the ET and may be seated along the cartilaginous part or only a portion thereof part of it; or it may be configured to be seated along the natural valve or escape it; or it may be configured for deployment to fill a larger portion of the length of the ET.

By some embodiments of this disclosure the stent may have a relatively uniform cross-sectional shape and dimension along its axis, in other embodiments the stent may have different cross-sections to fit different portions of the passageway; for example, in the case of an ET stent, proximal sections of the scaffold may be larger than distal ones.

The size (length and diameter) of body organs and also of passageways within the body, such as the ET, varies with age and accordingly a stent of this aspect may be designed to have different dimensions depending on its target population: for example, small stents used for infants, toddlers or young children and larger ones for older children or adults.

By one embodiment the ET stent has a scaffold that comprises an array of cells. The cells may be closed cells, open cells, or a combination of the two. The structural features of the cells influence the flexibility and displacement resistant properties of the scaffold. These include, for example, the relative proportion of open and closed cells, the overall size and configuration of cells, as well as the physical properties of the struts. For example, the relative proportion of open and closed cells may be different in different parts of the scaffold, imparting different physical properties (including flexibility and displacement resistance) to different portions of the scaffold. This may also be achieved by having cells of different sizes in different portions (larger cells generally causing the scaffold to be with increased flexibility and lower displacement resistance than portions with small cells). Larger cells or a high proportion of open cells may typically be at lateral portions of the scaffold.

By one embodiment the stent's scaffold is formed by generally zig-zagging, Z-shaped or sinusoidal-like struts that extend between the two opposite ends of the scaffold. The struts form oppositely oriented apexes. Consecutive apexes with the same orientation are separated from one another by an apex distance, referred to herein as apex distance; and consecutive opposite apexes are separated from one another by lateral distances, defined between the line tangential to the apexes, which will be referred to herein as amplitude length.

In some embodiments, opposite apexes of adjacent struts are circumferentially connected to form closed cells or some may not be connected to thereby form open cells. Different flexibility and displacement resistance of different portions of the stent may be achieved through variations in the properties and configurations of the struts which may be one or more of (i) variations in the apex distance; (ii) variations in the amplitude length; and (iii) variations in the struts in at least one portion of the scaffold as compared to at least one other portion.

By one embodiment of this disclosure, the stent has a scaffold which is constituted by two mirror-symmetric parts linked to one another only at the ends of the stent. This stent will be referred to below as a “mirror-symmetric stent”. While by some embodiments, the two parts of the mirror-symmetric stent may be articulated to one another at their two ends, they are typically integrally formed. The scaffold of the mirror-symmetric is also intrinsically biased into an expanded state that has said longitudinal plane of symmetry that extends between its two ends. Unlike conventional stents, which have many lateral links between different parts or portions of the scaffold, the two mirror-imaged parts of the scaffold of the mirror-symmetric stent are typically linked to one another only at their ends. This inherently configures the stent so as to have physical properties such that an inwardly directed lateral force, namely a force normal to the longitudinal plane of symmetry that axially extends between the two parts, causes an inwardly directed displacement; while a vertical force applied parallel to said plane, will cause no or very little displacement. Furthermore, this lateral displacement occurs without any distortions in other portions of the scaffold (which may happen upon inwards displacement of lateral portions in the case of a scaffold that constitutes a continuous circumferential structure).

The scaffold of the mirror-symmetric stent, has, typically, an overall non-circular cross-section with the two opposite mirror image parts usually defining an opposite generally vertical and, at times, outwardly-curved planes.

The mirror-symmetric stent may comprise two or more struts extending between the two ends, being linked to an integral with the mirror image struts in the opposite part of the scaffold. The struts may be linked to one another by lateral elements, e.g. lateral bars spanning the width between the struts.

The struts may also, by some embodiments, have an overall configuration similar to the struts described above with a generally sinusoidal zig-zagging or Z-shaped configuration, forming open or closed cells.

The stent is typically configured such that the scaffold is in its expanded state has an oversize of at least one portion of the scaffold than the corresponding portion of the lumen in which it is to be deployed. This oversize means that the stent would naturally expand to dimensions slightly larger than those of the lumen's corresponding portion. Such oversize causes the stent to apply constant force on the walls of the passageway and this fact, as well as oversize variations between different axial locations may aid in fixing the stent in position and avoid migration.

By some embodiments, the ET stent may comprise elements intended to aid in the stent removal. These elements may, for example, be integrally formed with the scaffold or may be non-integral elements linked or tied in some manner to the scaffold. Examples of such elements are a tailing arm formed by the joint ends of the struts of the scaffold that are braided together, may be a thread, cable, wire, suture or tab, at the proximal end.

By one embodiment, an arm, cable or tab attached to the proximal end of an ET stent may extend and protrude from the orifice of the ET into the Nasopharynx space. The arm, cable or tab may cross through the natural valve at the orifice of the ET and may engage with an anatomical feature, e.g. a muscle, that moves when swallowing. On such engagement, the arm, cable or tab will be pushed and apply pressure on the natural valve. The natural valve will be forced open and allow the ET to ventilate.

By some embodiments of this disclosure, the stent comprises anchoring elements integrally formed with the scaffold having the purpose of holding the stent in place to avoid its migration.

The stent of this disclosure may be made of a variety of materials, such as Nitinol, stainless steel, cobalt chromium, a variety of other metals, silicon rubber, a variety of polymeric materials particularly biodegradable or bio-absorbable materials such as poly-lactide base materials.

By other embodiments, the scaffold may be made of one material and coated by another, such as, for example, made from Nitinol struts that are coated by a polymeric material.

The stents of this disclosure may also be configured, by some embodiments, to elute drugs to their surrounding tissue. Drugs that may be included in such drug-eluting stents include, for example, steroids, anti-inflammatory drugs, antibiotics, etc. Techniques for incorporating drugs onto drugs are generally known.

The stents may be made by a variety of manufacturing techniques, such as laser cutting, braiding, 3D-printing, injection molding, compression molding, etc. By some embodiments, the stent may be configured

Provided by this disclosure, as also noted above, is a delivery system for deployment of the stent, such delivery system may include a delivery catheter, which in the case of an ET stent is inserted through the nasal orifice, preferably using a scope guidance. The delivery system may be based on such commonly used for delivering self-expanding stents and may include an outer sheath for compressing the stent to a small diameter and an inner sheath over which the stent is compressed. The delivery system may also comprise a guide wire that guides the advance of the inner sheath. Once in position the outer sheath may then be pulled back to expose the stent and allow it to deploy; and following deployment, the inner sheath may also be retracted. The catheter's manipulating end may be provide with markings to aid in safe deployment of the stent.

The stenting procedure of this disclosure may be preceded by a balloon expansion of the passageway, similar to that performed in angioplasty, intended to apply radial compression on the walls, expand the opening and squeeze out fluid and mucus. Such site preparation may make the stenting procedure easier and allow the stent to fully deploy inside the passageway to achieve full engagement with the surrounding tissue.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic representation of a stent of an embodiment of this disclosure deployed in the ET.

FIGS. 2A and 2B are views from the direction of arrow II in FIG. 1 in respective open and closed state of the ET.

FIG. 3 shows a stent of an embodiment of this disclosure having different dimensions in different portions along its length to fit corresponding portions of the ET and being configured such that its proximal end will jut out of the ET to permit stent removal.

FIG. 4 shows a stent configured for deployment in a specific portion of the ET, typically within the cartilaginous portion distal from the natural valve.

FIG. 5 shows a stent of this disclosure provided with a braided tail end.

FIG. 6 is a schematic representation of a stent with an annexed leg that extends into the nasopharynx cavity.

FIG. 7 is a schematic representation of an embodiment of mirror-symmetric stent.

### DETAILED DESCRIPTION OF EMBODIMENTS

The invention will now be further described with reference to some specific embodiments, schematically depicted in the annexed drawings. These embodiment concern ET stents but it should be understood that these embodiments are intended to illustrate and exemplify the teachings of this disclosure and in no way is it intended to be limiting; rather, they are examples of the broader teaching of this disclosure.

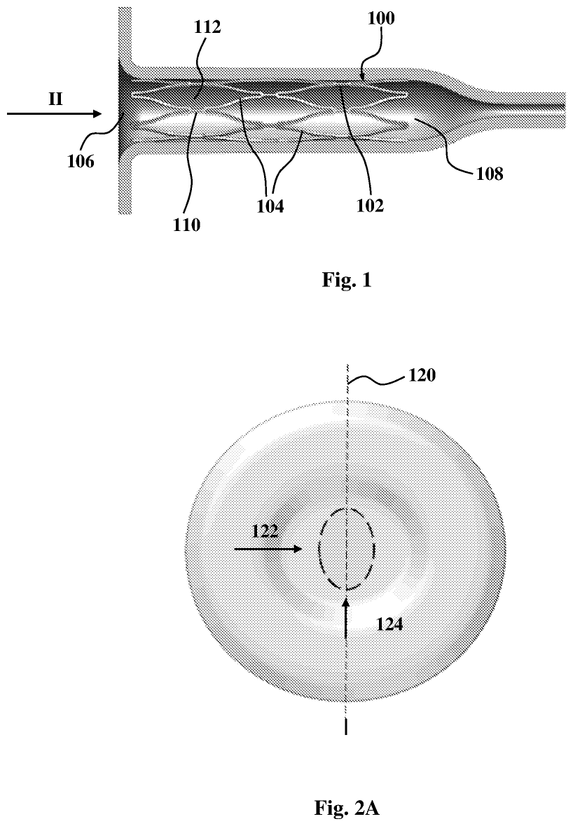

Reference is first being made to FIG. 1 showing a stent 100 according to an embodiment of this disclosure, deployed within the ET. Stent 100 has a peripheral scaffold 102 formed by a plurality of struts 104 that follow a generally sinusoidal path extending between the proximal end 106 and the distal end 108 of the stent. Opposite apexes, in adjacent struts of this sinusoidal structure, are connected to one another at connection points 110 to thereby define a plurality of closed cells 112.

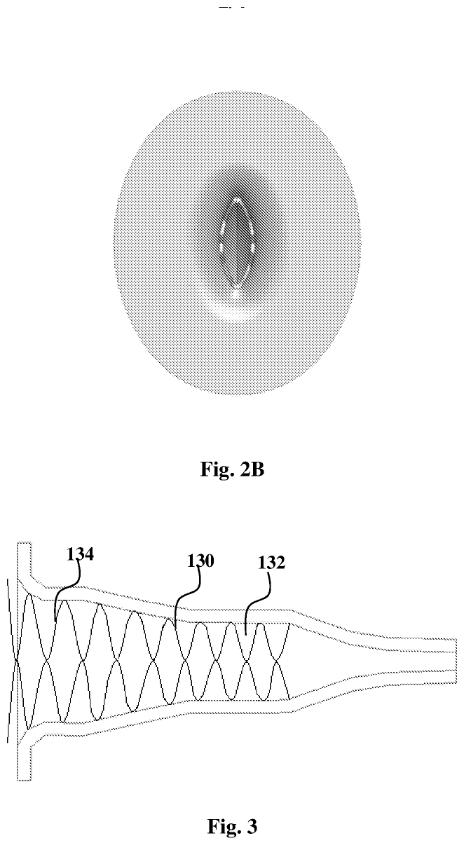

As can be seen in FIG. 2A the stent has an overall elliptical or oval cross-section defining a longitudinal plane of symmetry represented by dashed line 120, separating between the two sides that are mirror images of one another. The stent is thus configured such that an inwardly directed force in the direction normal to plane 120, as represented by arrow 122, would cause larger displacement than a similar force applied in a vertical inward direction, represented by arrow 124. Thus, when the walls of the proximal end of the ET close upon relaxation of the surrounding smooth muscles from the open state seen in FIG. 2A to the closed state seen in FIG. 2B, the two lateral walls displace inwardly permitting closure of the valve.

FIGS. 3-5 illustrate different stent configurations. In FIG. 3 stent 130 has segments with different cross-sectional dimensions including a distal segment 132 with a narrow dimension; and a proximal segment 134 with a wider dimension. The cells in segment 134 are larger and hence with larger apex distance and/or amplitude length and accordingly the rigidity and displacement resistance is overall lower than in the distal segment 132. There may also be variations in cell size, apex distance and amplitude length in upper and lower portions, as compared to lateral portions of the stent.

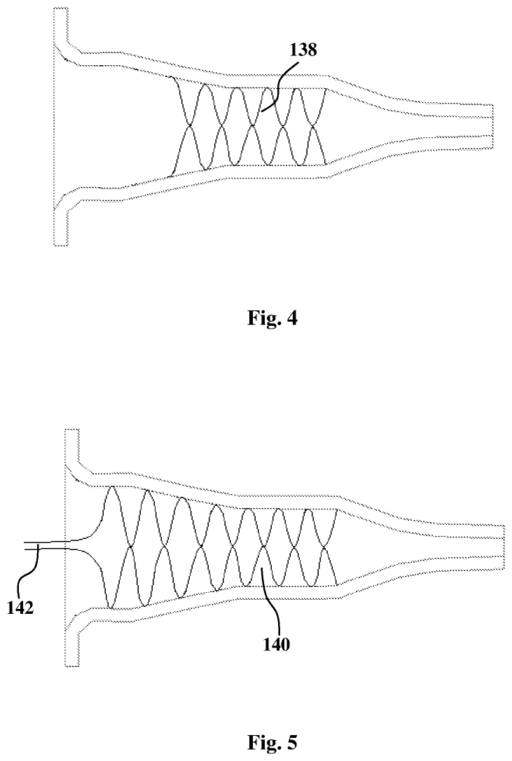

FIG. 4 illustrates a stent 138 configured for deployment in only a portion of the ET and FIG. 5 illustrates a stent 140 which has a tail end 142 constituted by the braided ends of the struts.

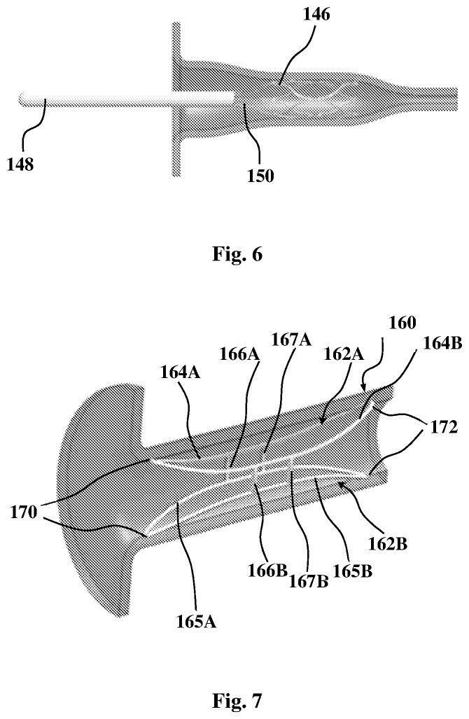

FIG. 6 illustrates a stent 146 with an arm 148 annexed to the proximal end 150 of the scaffold. The stent is deployed in an cartilaginous portion of the ET distal from the natural valve at the ET's proximal end and the arm 148, thus, crosses through the natural valve to engage with an anatomical feature, e.g. a muscle, that moves when swallowing. On such engagement, the arm 148 be pushed and apply pressure on the natural valve. The natural valve will be forced open and allow the ET to ventilate.

FIG. 7 illustrates a mirror-symmetric stent 160 that comprises two mirror-symmetric parts 162A and 162B extending lengthwise between the proximal and distal ends and comprising respective struts couples 164A, 165A and 164B, 165B, each integrated with its mirror image strut at integration points 170 and 172. The struts couples are linked to one another, by a pair of lateral bars 166A, 167A and 166B, 167B. Each of parts 162A and 162B form a generally vertical slightly outwardly-curved plane. This structure provides for flexibility of the scaffold only in the lateral direction about the terminal points of integration 170 and 172 and permitting very little, if any, inwardly-directed vertical displacement.

As cab be appreciated, the above stents are deployed such that the stents vertical portions juxtapose the lateral portions of the passageway and, consequently, permit a degree of lateral inward displacement of the walls of the.

The stent of this disclosure may be anchored by forces resulting from variations in radial force, cross-section and/or its eccentricity along its longitudinal axis.

The stent of this disclosure may also include means to hold the stent in place and avoid migration. Such means may comprise bars or other projections that protrude from the stent cylindrical envelope and anchor the stent in place. In case the stent is made by laser cutting, such bars or other projections may be formed at multiple locations along the stent length. Alternatively, zigzag pieces of the laser cut stent may be set to protrude from the stent cylindrical envelope (“fish scaling”) and help resist migration.

1-30. (canceled)

31. A stent with a peripheral scaffold being intrinsically biased into an expanded state with a length defined between a proximal and distal end of the scaffold; wherein
the scaffold has a longitudinal plane of symmetry extending between the two ends along the stent; and wherein
inwardly-directed force in a direction normal to the plane of symmetry causes an inwardly directed displacement that is larger than that caused by the same inwardly-directed force applied in a direction parallel to the plane of symmetry.

32. The stent of claim 31, wherein the scaffold is axial non-symmetric.

33. The stent of claim 32, wherein the scaffold has a non-circular cross-section and
is constituted by two mirror-symmetric parts linked to one another at both ends.

34. The stent of claim 31, wherein the scaffold comprises an array of cells, wherein the cells are one or a combination of closed and open cells and wherein the relative proportion of closed an open cells varies in different portions of the scaffold and wherein the cells in at least one portion of the scaffold are of different sizes than those of at least one other portion.

35. The stent of claim 31, wherein the scaffold is formed by generally zig-zagging struts extending between the two opposite ends with oppositely oriented apexes, consecutive apexes with the same orientation separated from one another by an apex distance and consecutive opposite apexes are separated by an amplitude length and
wherein the struts define a generally sinusoidal-shaped or Z-shaped curve.

36. The stent of claim 35, wherein opposite apexes of adjacent struts are circumferentially connected to define radial rings.

37. The stent of claim 35, wherein one or more of (i) the apex distance, (ii) the amplitude length and (iii) the strut's width in at least one portion of the scaffold is different than in at least one other portion.

38. The stent of claim 37, wherein the scaffold is configured to a larger displacement for a defined forces applied at the proximal end than at the distal end.

39. The stent of claim 31, wherein the scaffold has in its expanded state an oversize in at least one portion of the scaffold than the corresponding portion of the lumen in which it is to be deployed.

40. The stent of claim 31, comprising a tailing arm at the proximal end to aid in stent removal and a thread, cable, wire, suture or tab at the proximal end to aid in stent removal.

41. The stent of claim 31, comprising anchoring elements integral with the scaffold.

42. The stent of claim 31, for deployment in the Eustachian tube.

43. A stent with a peripheral scaffold being intrinsically biased into an expanded state with a length defined between a proximal and distal end of the scaffold; wherein
the scaffold has a longitudinal plane of symmetry extending between the two ends along the stent; and wherein
the scaffold is constituted by two mirror-symmetric parts linked to one another at both ends.

44. The stent of claim 43, wherein inwardly-directed force in a direction normal to the plane of symmetry causes an inwardly directed displacement that is larger than that caused by the same inwardly-directed force applied in a direction parallel to the plane of symmetry and optionally causes at a proximal portion of the scaffold causes an inwardly directed displacement that is larger than that caused by the same inwardly-directed force in a direction normal to the plane of symmetry at a more distal portion.

45. The stent of claim 43, wherein the scaffold has a non-circular cross-section and comprising an array of cells.

46. The stent of claim 43, wherein the scaffold is configured to a larger displacement for defined forces applied at different portions of the scaffold.

47. The stent of claim 43, wherein the scaffold has in its expanded state an oversize in at least one portion of the scaffold than the corresponding portion of a lumen in which it is to be deployed.

48. The stent of claim 43, wherein the proximal segment and the distal segment of the stent are mirror images of one another.

49. The stent of claim 43, comprising a tailing arm at the proximal end to aid in stent removal, a threads, cable, wire suture or tab at the proximal end to aid in stent removal and an anchoring elements integral with the scaffold.

50. A stent deployment system for deploying a stent of claim 31.
